(19) United States
(12) Patent Application Publication
(10) Pub. No.: US 2014/0326192 A1
(43) Pub. Date: Nov. 6, 2014

(54) ANTIMICROBIAL ANIMAL PRODUCT

(71) Applicants: COVENTRY UNIVERSITY, Coventry (GB); VALE BROTHERS LTD, Walsall, West Midlands (GB)

(72) Inventors: Stephen Coupe, Coventry (GB); Gracy Sailo-Lebeau, Coventry (GB)

(21) Appl. No.: 14/359,571

(22) PCT Filed: Nov. 30, 2012
(86) PCT No.: PCT/GB2012/052961

§ 371 Date: May 20, 2014

(30) Foreign Application Priority Data
Nov. 30, 2011 (GB) 1120613.3
Jan. 26, 2012 (GB) 1201279.5
Feb. 9, 2012 (GB) 1202291.9
May 15, 2012 (GB) 1208507.2

(51) Int. Cl. A01N 25/08; A01K 13/00; A01N 55/00

(52) U.S. Cl. CPC A01N 25/08; A01N 55/00; A01K 13/002
USPC 119/600; 424/404; 424/409; 514/63

(57) ABSTRACT

The invention provides an article for use with an animal, particularly a domesticated animal, comprising an antimicrobial agent and a polymer, wherein the antimicrobial agent is a quaternary ammonium compound. More specifically, the invention provides articles, such as grooming tools, for equestrian use which are capable of preventing the spread of diseases such as strangles, ringworm and diseases associated with Mycotoxins such as Fusarium.

This invention relates to antimicrobial polymers and polymer articles and their use in the manufacture of articles for use with animals, especially articles for use in stables or with horses (e.g. equestrian products) and articles for use with livestock or pets, and their use in the prevention of the spread of diseases such as strangles in horses.

Disease can spread quickly throughout groups of animals, particularly domesticated animals or those in captivity. In addition to the obvious animal welfare issues, it is often desirable to prevent the spread of disease among animals for economic reasons, e.g. in businesses such as stables and farms. In many cases, the spread of disease is due to micro-organisms such as bacteria.

For example, Strangles (Streptococcus equi subspecies equi infection) is an acute contagious upper respiratory tract disease, predominantly found in young horses and caused by the bacteria Streptococcus equi subsp. equi, which is a β-haemolytic group C Streptococcus. The symptoms include mucopurulent inflammation of the nasal passages, pharynx and associated lymph. Due to its contagious nature, the disease is subject to outbreaks which have both animal welfare and economic consequences. Moreover, Streptococcus equi subsp. equi has been reported to be involved in human infection, with cases of meningitis in humans being thought to be caused due to contact with infected horses.

Strangles can spread throughout stables via horses having direct contact with one another (e.g. rubbing noses) and indirectly due to sharing of drinking troughs, grooming equipment and other equipment such as saddles and the like. Vaccination is available, but has been found to have adverse effects and does not provide a consistently high level of protection (e.g. only around six months of immunity is achieved). Many horse owners are therefore reluctant to have their animal vaccinated as they do not consider the benefits to outweigh the risks. Current strategies for control of the spread of the disease are therefore limited to (i) monitoring horses for signs of sickness and/or testing; (ii) avoiding contact with other horses, especially those of unknown origin; (iii) limiting the numbers of horses present in a stable/yard in order to avoid overcrowding and (iv) isolating new horses for an initial period before introduction to the main group. Some of the above measures are not practical in a busy stable business and poor compliance with the measures can result in the spread of the disease.

There thus exists a need for means to control the spread of conditions such as strangles. Research has shown that S. equi can persist in dirty stables for several weeks and poor hygiene among horse handlers can exacerbate the spread of the disease via articles such as clothes, buckets, tack and grooming kit, e.g. brushes and combs.

It has been surprisingly found that articles for use with (e.g. in contact with) animals, including equestrian products such as horse grooming tools (e.g. brushes and combs), can be produced from polymers which have antimicrobial properties, e.g. polymeric materials that can inhibit the survival of micro-organisms responsible for diseases, such as certain bacteria. It is thus possible to produce equestrian products such as horse brushes and the like and products for use with other animals which have non-leaching antimicrobial properties and thus can reduce microbial cross-contamination, including strangles, by killing the microbe when it comes into contact with the active ingredient present in the brush.

Thus, viewed from a first aspect, the present invention provides an article for use with an animal, particularly a domesticated animal (e.g. a pet product) comprising an antimicrobial agent and a polymer. Viewed from a further aspect, the present invention provides an equestrian article comprising an antimicrobial agent and a polymer. Articles for use with household pets or livestock comprising an antimicrobial agent and a polymer are also encompassed.

While the features of the invention will now be described further in relation to equestrian articles, it should be understood that the present invention also encompasses equipment suitable for use with other animals, particularly domesticated animals or those in captivity, especially livestock/farm animals (e.g. cows, pigs, sheep, chickens, goats etc.) or household pets (e.g. cats, dogs, rabbits etc.), i.e. “livestock articles/products” and “pet articles/products”. The features of the invention herein described should be considered applicable to articles for use with any animal in a situation where cross-contamination with microbes may be an issue. Preferably the articles are of the type specially designed for use with animals. Many of the items described herein in relation to horses will be suitable for use with other animals. Such items also form part of the invention. Examples are blankets, rugs, buckets, troughs, bowls, grooming tools and the like.

Preferably the antimicrobial agent and the polymer are combined to form an antimicrobial polymer. As will be further described herein, the term “antimicrobial polymer” should be construed broadly and is thus intended to encompass a composition comprising, or consisting essentially of, an antimicrobial agent and a polymer. Preferably in the antimicrobial polymer the components are in intimate mixture with one another, e.g. the antimicrobial agent and the polymer are blended together or are copolymerised with one another. Particularly preferably the antimicrobial polymer is a blend of (i.e. consisting essentially of), or comprising, antimicrobial agent and polymer. In a preferred aspect of the present invention, the articles as herein described comprise a blend of antimicrobial agent and polymer.

Use of an antimicrobial agent and a polymer, e.g. an antimicrobial polymer, in the production of articles for use with animals, such as equestrian articles, e.g. horse brushes and the like, solves the problem of transfer of microbial infection from one animal to another. As the antimicrobial agent is not necessarily absorbed by the micro-organism, and is thus not required to leach from the polymer in order to be effective, it renders the substrate polymer antimicrobially active for extended periods of time.

Equestrian articles will be known and recognisable to those in the field, and the term is intended to cover all articles used in connection with horses in particular, but also donkeys. Equestrian articles should be understood to include items worn by horses, equestrian horse wear, horse grooming equipment/tools, stable equipment (e.g. yard utensils) and riding equipment.

Examples of items worn by horses are: horse rugs, horse blankets, saddle cloths, numnahs, leg wraps, bandages (e.g. knitted acrylic 3 m×10 cm, elasticated (3″ with tie or velcro, 4″ with tie or velcro) fleece 3.5 m×12 cm, woven, tail bandages), leg pads, horse boot liners, tail guards, travel boots, turnout boots, head and neck covers, fly veils, fly masks, poultice boots, girth liners, head collars (e.g. pony, cob, full and with or without cushioning), and matching reins.

Examples of horse grooming tools are: brushes (e.g. a grooming brush, a tail brush, a dandy brush, a body brush, a face grooming brush or a face finishing brush) combs, sponges, sweat scrapers, curry combs, plastic hoof picks, grooming bags, mane and tail brushes, mane combs, massage mitts, plaiting combs and stable cloths/rubbers.

Examples of stable equipment/yard utensils are: tack trays, tack boxes, buckets (flexible and hard), forks (e.g. shaving forks/fork handles), rakes, salt lick holders, bucket brushes, bucket covers, feeders, mangers, troughs, feed scoops, feed stirrers, plastic shovels, wheel barrows, saddle covers, brooms, haynets, haybags, fence post covers or wraps (e.g. flexible plastic wrap capable of taking a staple), stable floor mats, saddles, reins, horse toys such as boredom balls.

Examples of riding equipment are items worm by horse-riders such as: hat liners (internal), riding gloves, socks, jodhpurs, riding breeches and boot liners.

When the article is a horse grooming tool it is preferably a brush, a comb or a sponge, e.g. a grooming brush, a tail brush, a dandy brush, a body brush, a mane brush, a curry comb, a face grooming brush or a face finishing brush. Viewed from a further aspect the invention provides a horse grooming tool comprising an antimicrobial agent and a polymer.

Other preferred equestrian articles are scoops, buckets, troughs, blankets and rugs.

The article of the invention comprises an antimicrobial agent and a polymer. All or part (e.g. the part most frequently in contact with the animal) of the article may comprise an antimicrobial agent and a polymer, e.g. all or part of the article may be coated with an antimicrobial agent and a polymer.

More preferably, the article of the invention may be made entirely from antimicrobial polymer, or only part of the article (e.g. the bristles of a brush) may be formed from antimicrobial polymer. Alternatively, the article may be entirely or partially coated with antimicrobial agent or antimicrobial polymer according to the invention. When only part of the article is formed from the antimicrobial polymer, or coated with antimicrobial agent or antimicrobial polymer, that part should preferably be the part that is most frequently in contact with the animal during use, e.g. the bristles of a brush, or the teeth in the case of a comb. Typically, at least the part most usually in contact with the animal (e.g. brush bristles or comb teeth) is formed from the antimicrobial polymer, or is coated with antimicrobial agent or antimicrobial polymer. Particularly preferably, the article is made entirely from, or is entirely coated with, the antimicrobial polymer. Especially preferably the entire article (or the part most usually in contact with the animal) is made from the antimicrobial polymer.

The article of the invention may be entirely or partially formed from a textile, or entirely or partially coated with, or covered by, a textile, wherein the textile comprises an antimicrobial agent and a polymer, e.g. an antimicrobial polymer, as herein described. The textile may be a conventional textile which has been coated with antimicrobial agent or antimicrobial polymer, but more preferably it is woven using fibres comprising, or consisting essentially of, antimicrobial polymer. The fibres comprising, or consisting essentially of, antimicrobial polymer may be used alone or in combination with another type of fibre to make (e.g. weave or knit) a textile with antimicrobial properties. The fibres comprising antimicrobial agent may be combined (e.g. spun) with other fibres before knitting or weaving, and/or may be combined with other fibres via knitting or weaving. Where the antimicrobial polymer fibres are used in combination with another fibre, the other fibre is typically a conventional material for making textiles, e.g. a synthetic material such as nylon, polyester, acrylic, polypropylene or a natural material such as wool, silk, cotton. In certain embodiments, the article of the invention is made from, or comprises, a textile woven from fibres of antimicrobial polymer as herein described. Such textiles, comprising an antimicrobial agent and a polymer as herein described in relation to articles, form a further aspect of the present invention.

The article of the invention is imparted with antimicrobial properties by combining an antimicrobial agent with a polymer. Preferably this is achieved by incorporating an antimicrobial agent into or with the polymer.

Particularly suitable antimicrobial agents are those which are capable of killing microbes without leaching from the polymer. Because the active agent in this case does not leach from the polymer during use and thus the animal is not at risk from contact with the chemical, this may be termed “non-chemical killing” of the microbes. The fact that the active agent does not leach, renders the polymer/antimicrobial combination (e.g. the antimicrobial polymer as herein described), and thus the article, more stable and longer lasting. The compositions used in the present invention have thus been found to be both stable and effective.

Preferred antimicrobial agents for use in the invention are therefore those which are capable of affecting microbial cells (i.e. interacting at a molecular level) without actually having to leach out of the polymer and come into contact with the host animal (e.g. the horse). Such means of interaction include enzyme inactivation and cell disruption, e.g. outer cell disruption, or disruption of intermolecular interactions such as dissociation of cellular membrane lipid layers. This interaction is typically electrostatic, thus antimicrobial agents which contain, produce or involve in their method of action, charged species are particularly preferred.

Preferred antimicrobial agents include silver compounds, biguanides (also known as biguanidines), epoxy compounds, quaternary ammonium compounds (“quats”, preferably “Si-quats”).

Examples of suitable silver compounds are silver salts/ions, e.g. AgNO₃, silver zeolites, silver sulphadiazine (AgSD), silver acetate, silver protein etc. Metallic silver may also be used.

Silver ion-based antimicrobials such as silver nitrate work against microbes such as bacteria and fungi via the interaction of Ag+ ions with microbial thiol groups, particularly in enzymes and proteins. The silver works as a catalyst for the oxidation reactions which denature the sulphide bonds in the proteins of bacteria. Inactivation of enzymes leads to a loss of internal control by the organism. Silver ions may also bind to bacterial cells, thereby altering the cell membrane function. A release of potassium ions may also be facilitated by Ag+, decreasing membrane function. Inhibition of growth of microbes has been reported when using Ag+ releasing compounds, as has disruption of membranes. The deposition of granules of silver nitrate within cell walls and vacuoles has also been reported.

The antimicrobial action of silver sulphadiazine (AgSD), is thought to be due to both components of the compound, i.e. both the SD and Ag+.

Silver compounds have the advantage that very few bacteria are intrinsically resistant. Moreover, the active Ag+ ion is not toxic to human cells and silver compounds are able to act as long lasting biocides with high temperature stability and low volatility.

Silver compounds may be combined with the polymer in the present invention by the means described herein for other antimicrobial agents. In particular, metallic silver may be deposited directly onto the surface of a polymer substrate using methods such as vapour coating, sputter coating, ion beam coating, deposition or electrochemical deposition of silver from solution. Silver may also be incorporated into a polymer by mixing into molten polymer, or by co-extrusion of a silver compound (e.g. a silver zeolite) with the polymer. Silver-containing (e.g. silver-coated or silver-incorporated) polymers may be used to produce fabrics with antimicrobial properties.

Examples of suitable biguanides are compounds of the following formula, salts, analogues and/or polymers thereof in which each R group is independently selected from an organic group, hydrogen or a hydroxy group. By “organic group” is meant a group containing one or more carbon atoms, preferably a group consisting essentially of carbon atoms and hydrogen atoms, optionally further comprising one or more heteroatoms (e.g. N, O, Si, P, S, etc.):

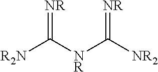

Preferred R groups are hydrogen, alkyl, aryl or alkaryl groups, including alkoxy and aryloxy groups and hydrocarbon chains, e.g. C₂₋₂₀ alkyl chains, preferably C₄₋₁₀ alkyl chains, for example propyl or hexyl chains.

Compounds comprising two or more biguanide groups are preferred, particularly those in which the biguanide groups are linked by alkyl, aryl or alkaryl groups, including alkoxy and aryloxy groups. Preferably the biguanide groups are linked by a hydrocarbon chain, e.g. a C₂₋₂₀ alkyl chain, preferably a C₄₋₁₀ alkyl chain, for example a propyl or hexyl chain. Polymeric forms of such compounds are especially preferred and examples are polyhexamethylene biguanide (PHMB) and polyaminopropyl biguanide (PAPB) and salts thereof, e.g. polyhexamethylene biguanide chloride, shown below.

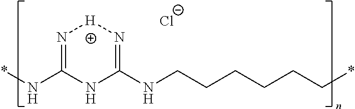

The mode of action of biguanides involves outer cell disruption which often sub lethally damages the microbe. The inner membrane of the microbe is then damaged leading to cell leakage and the coagulation of the inner cell constituents in some cases.

Biguanide antimicrobials prevent spore development and are effective against most bacteria, yeasts, protists (the ‘animal like’ single celled microbes including the amoeba) e.g. Cryptosporidium, Entamoebae, the malarial parasite Plasmodium and some viruses.

PHMB and PAPB are specifically bactericidal at very low concentrations and are also fungicidal. PHMB is not cytotoxic, can be directly applied to wounds and does not cause irritation like other traditional disinfectants such as alcohol. The polymer strands are incorporated into the bacterial cell wall, which then disrupts the membrane and reduces permeability. It binds to the bacterial DNA, alters its functions and causes lethal DNA damage.

Preferred antimicrobial agents are quaternary ammonium compounds (“quats”), preferably (“Si-quats”). Quaternary ammonium compounds have a range of uses, e.g. as disinfectants, surfactants, fabric softeners, and as antistatic agents. Their antimicrobial activity lends them to application as disinfectants and sanitizing agents. Examples, which may be used in the invention, are benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride and domiphen bromide. Where one of more of the organic groups comprises silicon, the compound is known as a “Si-quat”.

Preferably the antimicrobial agent is a quaternary ammonium compound, e.g. a compound of general formula (I) or a polymer (e.g. a homopolymer) thereof:

[R₄N]⁺X⁻ (I)

where each R is independently selected from an organic group, hydrogen or a hydroxy group and X⁻ is an anion, typically halide, hydroxyl, acetate, SO₄²⁻, CO₃²⁻ or PO₄³⁻, preferably a halide. By “organic group” is meant a group containing one or more carbon atoms, preferably a group consisting essentially of carbon atoms and hydrogen atoms, optionally further comprising one or more heteroatoms (e.g. N, O, Si, P, S, etc.). The R groups are typically independently selected from hydroxy, hydrogen, alkyl, aryl or alkaryl groups, including alkoxy and aryloxy groups. Preferably the R groups have 1 to 30 carbon atoms, especially 1 to 20, e.g. 2 to 12. The R groups may be branched or linear and may contain heteroatoms either in the chain/ring or as part of a substituent. Preferably at least one of the R groups is a long chain alkyl group, e.g. a C₁₂ to C₂₀ alkyl, especially a C₁₄ to C₁₈ alkyl group.

Especially preferably, the R groups of formula (I) are independently selected from short chain alkyl groups, such as C₁₋₆ alkyl groups (e.g. methyl); C₇₋₂₀ alkyl groups, (e.g. C₁₂₋₁₄ alkyl groups) and aralkyl groups, such as benzyl groups.

Especially preferably, each R in formula (I) is an organic group and X⁻ is an anion, typically halide, hydroxyl, acetate, SO₄²⁻, CO₃²⁻ or PO₄³⁻, preferably a halide. By “organic group” is meant a group containing one or more carbon atoms, preferably a group consisting essentially of carbon atoms and hydrogen atoms, optionally further comprising one or more heteroatoms (e.g. N, O, Si, P, S, etc.). The R groups are typically independently selected from alkyl or aryl groups, including alkoxy and aryloxy groups. Preferably the R groups have 1 to 30 carbon atoms, especially 1 to 20, e.g. 2 to 12. The R groups may be branched or linear and may contain heteroatoms either in the chain/ring or as part of a substituent. Preferably at least one of the R groups is a long chain alkyl group, e.g. a C₁₂ to C₂₀ alkyl, especially a C₁₄ to C₁₈ alkyl group.

Further preferred compounds of formula (I) are those in which one or more of the R groups are hydrogen or hydroxy groups.

Especially preferably, two of the R groups are short chain alkyl groups, such as C₁₋₆ alkyl groups (e.g. methyl) and two of the R groups are independently selected from alkyl groups, preferably C₇₋₂₀ alkyl groups (e.g. C₁₂₋₁₄ alkyl groups) and aralkyl groups, such as benzyl groups.

Particularly preferred quaternary ammonium compounds are benzalkonium chloride (BAC) and didecyldimethyl ammonium chloride (DDAC), e.g. compounds of the following formulae:

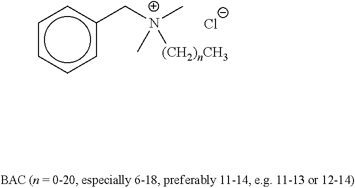

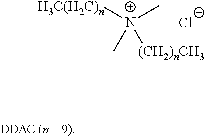

Benzalkonium salts are active against bacteria, some viruses, fungi and protozoa. The solutions can be bacteriostatic or bactericidal depending on the concentration used. Gram positive bacteria are more susceptible than Gram negative bacteria. Benzalkonium chloride is readily soluble in ethanol and acetone and dissolves slowly in water. It is a rapidly acting biocidal agent with a moderately long duration of action. The mechanism of bactericidal or microbiocidal action is due to disruption of intermolecular interactions which can occur at several sites in the cell resulting especially in dissociation of cellular membrane lipid bilayers. When this happens, the permeability control of the membrane is lost and results in leakage of the cell contents. Other cellular contents can also undergo changes especially enzymes that control cellular activities. As it is also a cationic surfactant, critical intermolecular activities can be disrupted.

DDAC shares its overall range of properties with BAC. It has been reported to have no unacceptable effect on environments and that the residual effect is not significant or harmful to human and animals. DDAC is a registered biocide for control of algae, bacteria, fungi or molluscs.

Especially preferably, the antimicrobial agent is a compound of formula (II) or a polymer (e.g. a homopolymer) thereof

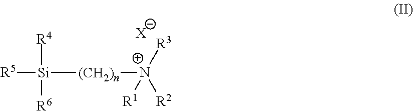

where X⁻ is an anion, e.g. a halide (i.e. F⁻, Cl⁻, Br⁻, I⁻), hydroxyl, acetate, SO₄²⁻, CO₃²⁻ or PO₄³⁻, preferably a halide, especially preferably Cl⁻;

n is an integer from 0 upwards, e.g. from 0 to 20, preferably 1 to 12, especially preferably 2 to 6, particularly preferably 3;

R¹ to R⁶ are independently selected from an organic group, hydrogen or a hydroxy group, preferably hydroxy, hydrogen, alkyl, aryl or alkaryl groups, including alkoxy and aryloxy groups. By “organic group” is meant a group containing one or more carbon atoms, preferably a group consisting essentially of carbon atoms and hydrogen atoms, optionally further comprising one or more heteroatoms (e.g. N, O, Si, P, S, etc.). Preferably the R groups have 1 to 30 carbon atoms, especially 1 to 20, e.g. 2 to 12. The R groups may be branched or linear and may contain heteroatoms either in the chain/ring or as part of a substituent.

The R groups of formula (II) may be independently selected from short chain alkyl groups, such as C₁₋₆ alkyl groups (e.g. methyl); C₇₋₂₀ alkyl groups (e.g. C₁₂₋₁₄ alkyl groups) and aralkyl groups, such as benzyl groups.

Preferably at least one, e.g. one, two or three, particularly one or two, of R¹, R² and R³ is a long chain alkyl group, e.g. a C₁₂ to C₂₀ alkyl, especially a C₁₄ to C₁₈ alkyl group.

Preferably at least one, e.g., one, two or three of R⁴, R⁵ and R⁶ are independently selected from an alkoxy group, e.g. propoxy, ethoxy or methoxy, preferably a methoxy group.

In one aspect, R¹ to R⁶ of formula (II) are independently selected from alkyl or aryl groups, including alkoxy and aryloxy groups. Preferably the R groups have 1 to 30 carbon atoms, especially 1 to 20, e.g. 2 to 12. The R groups may be branched or linear and may contain heteroatoms either in the chain/ring or as part of a substituent.

Preferably at least one, e.g. one, two or three, particularly one or two, of R¹, R² and R³ is a long chain alkyl group, e.g. a C₁₂ to C₂₀ alkyl, especially a C₁₄ to C₁₈ alkyl group.

Preferably at least one, e.g., one, two or three of R⁴, R⁵ and R⁶ are independently selected from an alkoxy group, e.g. propoxy, ethoxy or methoxy, preferably a methoxy group.

Further preferred compounds of formula (II) are those in which one or more of the R groups, especially one or more of R⁴, R⁵ and R⁶ are hydrogen or hydroxy groups.

Particularly preferably, the antimicrobial agent is 3-(trihydroxysilyl)propyldimethyl octodecyl ammonium chloride or a polymer (e.g. a homopolymer) thereof, or 3-(trimethoxysilyl)propyldimethyl octodecyl ammonium chloride i.e. a compound of formula (III) or a polymer (e.g. a homopolymer) thereof:

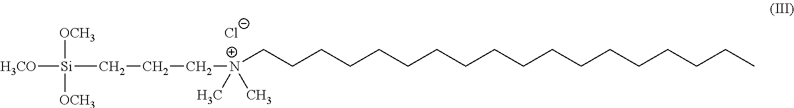 [SMILES: [Cl-] ; CCCCCCCCCCCCCCCCCC[N+](C)(C)CCC[Si](OC)(OC)OC]

3-(trihydroxysilyl)propyldimethyl octodecyl ammonium chloride (shown below as a compound of formula (IV))

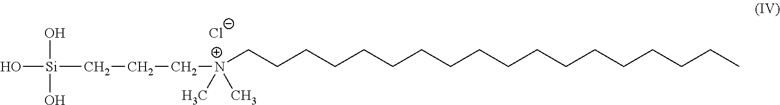 [SMILES: [Cl-] ; CCCCCCCCCCCCCCCCCC[N+](C)(C)CCC[Si](O)(O)O]

is available from BIOSAFE Inc. under the name “HM4100” as a polymeric solid cationic quaternary ammonium salt antimicrobial agent. HM4100 is in the form of a crystalline powder that is thermally stable in injection moulding and extrusion. It is effective in lower concentrations than typical for antimicrobials.

Compounds related to those of formulae (III) and (IV) are also preferred as the antimicrobial of the invention. Examples are those in which the C₁₈ tail is replaced by an alternative long chain alkyl group, e.g. C₁₂ to C₂₀ alkyl group. Compounds which differ from those of formulae (III) and (IV) in that they comprise a —(CH₂)—₂₋₁₂ linker, preferably a —(CH₂)—₂₋₆ linker, between the nitrogen and silicon atoms are also preferred as the antimicrobial of the invention.

The antimicrobial agent may be combined with any polymer suitable for making the articles of the invention, e.g. elastomers, silicones, thermoplastics, particularly a thermoplastic. Preferred polymers are polyurethanes, nylon, polyolefins (such as polypropylene (PP), polyethylene (PE), high density polyethylene (HDPE), low density polyethylene (LDPE), linear low density polyethylene (LLDPE)), polyesters, polyethylene terephthalate (PET), polyvinyl chloride (PVC), polystyrene (PS), high impact polystyrene (HIPS), polyamides (PA), acrylonitrile butadiene styrene (ABS), melamine formaldehyde (MF), polycarbonate (PC), elastomers (such as thermoplastic elastomer (TPE)), polysiloxanes/silicones (e.g. silicone (SI)) and polycarbonate/acrylonitrile butadiene styrene (PC/ABS), Petex, derivatives, mixtures and blends thereof. Especially preferably the polymer is a polyolefin, e.g. polypropylene, or a polymer blend comprising polypropylene.

The antimicrobial agent may be employed in monomeric form, polymeric form (e.g. as a homopolymer of the antimicrobial compound) or may be co-polymerised with the polymer of the invention.

The antimicrobial agent and the polymer may be combined in a variety of ways, e.g. a polymer article may have a coating of, or comprising, antimicrobial agent applied thereto, or the antimicrobial agent may be mixed with, e.g. incorporated into, the polymer using techniques known in the art (e.g. extrusion). Preferably the antimicrobial agent is incorporated into the polymer, e.g. by blending, dissolution or copolymerisation.

The polymer and antimicrobial agent can be combined to form an antimicrobial polymer in a variety of ways. Antimicrobial polymers according to the invention can be produced by the following processes:

(i) Polymer in solid form (e.g. particles such as powder, beads, pellets, granules or flakes) is coated with a solution or melt of the antimicrobial agent (which may be, but is not necessarily, in polymer or polymerisable form) to create coated solid polymer particles. The thus coated polymer may be ready to use, or in concentrate form, i.e. a concentrated “master-batch” for future blending (e.g. by melting and/or extrusion) with more polymer to produce a resin with antibacterial properties. The coating of the solid polymer particles is preferably carried out by spraying the polymer particles with the solution of the antimicrobial agent. The coated particles are then preferably dried (e.g. to remove solvent) before further use. Alternatively, polymer can be mixed with antimicrobial agent in liquid form by means other than by coating polymer particles, e.g. polymer in solid or liquid form may be mixed with antimicrobial agent in liquid form, with or without heating, e.g. the polymer particles may be mixed (e.g. by stirring) in a solution of antimicrobial agent.

(ii) A polymerisable antimicrobial monomer (e.g. in solid, liquid, solution or melt form) is copolymerised with a second monomer and/or a polymer to produce a copolymer of the antimicrobial agent with the second monomer and/or the polymer.

(iii) A polymerisable antimicrobial monomer (e.g. in solid, liquid, solution or melt form) is blended with a second monomer and/or a polymer to produce a homopolymer of antimicrobial agent blended with the second monomer and/or the polymer. In this way a blended polymer comprising a homopolymer of antimicrobial agent is formed.

(iv) Antimicrobial agent (which may be, but is not necessarily, a polymer or polymerisable) in solid form (e.g. particles such as powder, beads, pellets, granules or flakes) is blended with polymer in solid form (e.g. particles such as powder, beads, pellets, granules or flakes), e.g. by melting, mixing and/or extrusion.

(v) Particles comprising antimicrobial agent and first polymer (e.g. a master-batch as produced by (i) above) are blended with a second polymer (which may be the same or different to the first polymer) in solid form, e.g. by melting or extrusion.

(vi) A solution of antimicrobial agent in a solvent (or antimicrobial agent in liquid, e.g. melt form) is mixed with polymer in solid form (e.g. particles such as powder, beads, pellets, granules or flakes). Preferably the particles are then dried in order to remove solvent from the pellets.

(vii) Antimicrobial agent (which may be, but is not necessarily, a polymer or polymerisable) in liquid form (e.g. as a solution or melt) is blended with polymer in solid or liquid form (e.g. a melt, a liquid, a solution, or particles such as powder, beads, pellets or flakes), e.g. by melting, mixing and/or extrusion.

(viii) Antimicrobial agent (which may be, but is not necessarily, a polymer or polymerisable) in solid form (e.g. as powder, beads, pellets, granules or flakes) is blended with polymer in solid or liquid form (e.g. a melt, a liquid, a solution, or particles such as powder, beads, pellets or flakes), e.g. by melting, mixing and/or extrusion.

When a solution of antimicrobial compound is required, the solvent is preferably a polar solvent, especially preferably an alcohol, particularly, methanol, ethanol, butanol, propanol (e.g. isopropyl alcohol).

The above-mentioned second monomer may be the same or different to any one of the monomers that form the main polymer of the composition.

The present invention thus provides articles as herein described (e.g. horse grooming tools) comprising an antimicrobial polymer, wherein said polymer is, i.e. consists essentially of, or comprises one or more of the following:

(a) a copolymer of an antimicrobial monomer and a second monomer;

(b) a copolymer of an antimicrobial monomer and a polymer;

(c) a copolymer of an antimicrobial monomer, a second monomer and a polymer;

(d) a blend of a homopolymer of antimicrobial agent and a second monomer;

(e) a blend of a homopolymer of antimicrobial agent and a polymer;

(f) a blend of a homopolymer of antimicrobial agent, a second monomer and a polymer;

(g) a mixture of an antimicrobial compound and a second monomer and/or a polymer, and

(h) solid polymer coated with antimicrobial compound (which may be polymerisable or a polymer)

The antimicrobial polymers herein described can be used to form the articles of the invention, or can be used (e.g. in liquid, i.e. solution or melt form) to coat a pre-prepared article, e.g. by spraying or slurrying. Alternatively, the polymer article may be coated with antibacterial compound.

In a particularly preferred aspect, a concentrated “master-batch” of antimicrobial agent in polymer is prepared, for example by one of the methods described above, for later combination with polymer (in e.g. melt form) to produce a blend of the desired concentration of antimicrobial agent in polymer. A typical master-batch composition has an antimicrobial agent content (expressed as a weight percentage of the master-batch) of 2 to 75%, e.g. 3 to 50%, preferably 4 to 25%, especially preferably 5 to 10%. In this aspect, the polymer in solid form such as powder, beads, pellets or flakes may be coated (e.g. by spraying or mixing) with a solution or melt of the antimicrobial agent to create a coated solid polymer concentrate for future blending to produce a resin with antibacterial properties. Alternatively, the master-batch may be formed by mixing antimicrobial agent in solid form with polymer in solid form, e.g. by extrusion.

Alternatively, the antimicrobial agent (e.g. in liquid or granular form) may be added to a melt of the polymer either alone or in a solution at the desired concentration and the melt mixed to dissolve the antimicrobial agent in the polymer melt.

The mixing of the components is effected using typical polymer processing techniques, e.g. melting, mixing and/or extrusion. In general no chemicals are required for processing. In one aspect, the antimicrobial agent is added (preferably in liquid or granular form) to the polymer. The components are then mixed, optionally with heating, e.g. to melt one or both components. Particularly preferably, the antimicrobial agent and polymer in solid form (and optionally, any other components which are desired in the polymer composition) are extruded (e.g. using a single or twin screw extruder) in a process where the material will be fused to form pellets. Fusion typically takes place at temperatures between 150 and 280° C., preferably between 180° and 240° C. Heating to these temperatures before mixing and extrusion is preferred. The product (e.g. pellets) produced may optionally then be blended (e.g. by melting) with more polymer before being further processed to form articles according to the invention, e.g. by extrusion through a die to form filaments which may be used as bristles of a brush, or threads for weaving into a textile.

The amount of antimicrobial agent in the antimicrobial polymer is typically less than 10 wt % (expressed as a percentage of the resulting antimicrobial polymer), especially less than 5 wt %, e.g. 0.1 to 2.5 wt %, preferably 0.25 to 2 wt %, especially 0.5 to 1.5 wt %, particularly preferably around 1 wt % or 2 wt %. In aspects of the invention where the antimicrobial agent and the polymer are both present in the article but are not necessarily combined to form an antimicrobial polymer, the amount of antimicrobial agent, with reference to the total weight of antimicrobial agent and polymer combined, is typically less than 10 wt %, especially less than 5 wt %, e.g. 0.1 to 2.5 wt %, preferably 0.25 to 2 wt %, especially 0.5 to 1.5 wt %, particularly preferably around 1 wt % or 2 wt %.

Other components may be present in the polymer or antimicrobial polymer of the invention. The further components, when present, may be selected from colourants (e.g. dyes), UV inhibitors, surfactants, chain transfer agents and other polymerisation modifiers, cross-linking agents, plasticizers, polymerisation modifiers, property modifiers, stabilizers and mixtures thereof. These additives may be present in typical amounts and may be added at a suitable stage of processing, e.g. when the antimicrobial agent and a polymer are combined, or at a later stage, e.g. when a concentrated “master-batch” comprising antimicrobial agent and polymer is mixed with further polymer.

The articles of the invention can be made by conventional means. When the entire article is formed from polymer, the composition comprising antimicrobial agent and polymer can be moulded into the shape of the tool using conventional techniques such as extrusion moulding, extrusion blow moulding, rotational moulding, injection moulding or injection blow moulding. Extrusion may be effected, for example, using a twin or single screw extruder. Alternatively, part of the article may be formed from the composition comprising antimicrobial agent and polymer, in which case that part is made as described above with reference to an entire article.

Where the article comprises, is formed from, or is coated/covered with textile, the textile is woven from fibres containing antimicrobial agent and polymer or is woven using fibres of antimicrobial polymer (optionally in combination with other types of fibres). The fibres (e.g. of antimicrobial polymer) are formed from compositions as herein described, i.e. a composition comprising antimicrobial agent and polymer (e.g. an antimicrobial polymer as herein described) by conventional means for formation of synthetic fibres, e.g. by extrusion. Typical fibre diameters are 0.05 to 2 mm, especially 0.1 to 1.2 mm, e.g. around 0.45 mm. Fibres can be formed of any practical length, depending on their intended use. For brushes and the like, lengths of 20 to 250 mm, preferably 50 to 200 mm are suitable. Particularly, lengths of around, 64, 70, 100 and 170 mm are suitable for brush bristles. For manufacture of textiles and fabrics, longer fibres may be required, e.g. up to several thousands of metres. Threads, fibres and yarns may be mono-filaments or made by twisting, otherwise bonding or simply grouping together a number of separate fibre strands to form the fibre/thread/yarn to be used to produce a textile. As noted above, fibres comprising antimicrobial agent and polymer may be combined in these ways with other types of natural or synthetic fibres. Similar methods may be used to make fibres for other uses, e.g. as brush bristles. Fibres of any desired cross-section may be produced using convention techniques such as extrusion. Typical fibre cross-sections for use in the invention are circular, flat, and x-shaped.

Antimicrobial agent may be present in the article either by virtue of being combined with the polymer prior to the production of the tool, or by coating the formed article with antimicrobial agent. Alternatively, only the parts of the article that routinely come into contact with the animal is formed from, or coated with, the polymer (e.g. the antimicrobial polymer). In this case, that part, e.g. brush bristles, comb teeth or sponge parts is typically formed from the antimicrobial polymer (or formed from polymer and then coated with antimicrobial agent) and is attached to the rest of the tool, e.g. a handle or base.

The brush fibres can be made by mixing granules of antimicrobial agent with polymer before melting and extruding the mixture to form fibres, of varying diameters, e.g. from 0.1 mm to 1.2 mm. The fibres are typically then bundled together and the bundles are cut to the lengths needed to produce brush bristles, e.g. 64 or 70 mm for the fibres of a thinner diameter, and 100 mm and 170 mm length with fibres that are around 0.45 mm in diameter.

For example, brush bristles may be formed by combining 3-(trihydroxysilyl)propyldimethyl octodecyl ammonium chloride with polypropylene stock at a dosing rate of around 1% or 2% by total weight and the mixture melted, before being extruded into fibres that can be incorporated into a brush as the brush's bristles.

In order to form the brush, holes are typically drilled or moulded into brush backs, which can be made of wood, plastic or leatherboard, or any similar material. The fibres of antimicrobial polymer can then be folded around a piece of wire which is cut to size and hammered into the hole. Methods of securing the fibre include (i) using a staple to hold the fibre in the drilled hole—the staple goes into the hole and secures into the brush back at the base of the hole, and (ii) cutting or preforming the wire into a straight bar that wedges across the hole entrance to hold the fibre in place.

In the case where only part of an article needs to contain antimicrobial agent, that part can be formed in a similar method to the brush bristles above. For example, a sweat scraper for horses can be made by blending a Si-quat of formula (II) such as a homopolymer of 3-(trihydroxysilyl)propyldimethyl octodecyl ammonium chloride with a polymer such as polypropylene at 0.5-2 wt %. The components are blended with heating and moulded into the required shape for a sweat scraper blade using means convention for the production of such articles, e.g. extrusion moulding, extrusion blow moulding, rotational moulding, injection moulding or injection blow moulding. A conventional handle can then be attached to the blade. This method of manufacture could also apply to items where only one side is routinely in contact with the animal, e.g. rugs and blankets with distinct sides, items of clothing and items with handles (the handles may not require antimicrobial agent). The part which comes into contact with the animal most frequently can be made to contain antimicrobial agent and polymer according to the invention and the rest of the article may be made conventionally.

Similarly, items which are to be formed entirely from antimicrobial polymer can be moulded after the components are mixed. For example, a bucket can be formed by moulding antimicrobial polymer of the invention into the required shape using conventional techniques, e.g. extrusion moulding, extrusion blow moulding, rotational moulding, injection moulding or injection blow moulding. The antimicrobial polymer is typically formed by blending polypropylene with an antimicrobial such as a homopolymer of 3-(trihydroxysilyl)propyldimethyl octodecyl ammonium chloride at around 1% wt or 2% wt. Similar methods can be used for the production of combs, mats, bowls, toys and the like.

In order to produce a textile comprising an antimicrobial agent and a polymer, fibres/threads of antimicrobial polymer may be made by forming the antimicrobial polymer into threads/fibres (e.g. by extrusion) suitable for weaving or knitting into a fabric. If necessary, the fibres may be spun prior to being woven into the textile. The fibres of antimicrobial polymer may be combined with conventional fibres as described above, e.g. by weaving the fabric from both types of fibre, or by spinning antimicrobial fibres and conventional fibres together prior to weaving the textile. Such textiles may be used to line items of clothing, or can be used as bandages, blankets, rugs etc.

Alternatively, articles can first be produced and then treated to impart antimicrobial properties. For example, articles comprising textile, e.g. rugs or blankets, may first be produced (by conventional methods such as weaving etc.) and then treated to give them antimicrobial properties. Such articles may be woven from polymer fibres and then coated with antimicrobial agent or antimicrobial polymer, or may be woven from a natural or non-polymeric material and then coated with antimicrobial agent or antimicrobial polymer.

The articles of the invention are resistant to micro-organisms either by the antimicrobial agent being present with or within the polymer or as a coating on the article. In addition to preventing the spread of strangles by killing or inhibiting the growth of Streptococcus equi subsp. equi (e.g. Streptococcus equi subsp. equi NCTC 9682) bacteria which are transferred from an infected horse to the article, the antimicrobial polymer compositions of the invention can be used to control the spread of other organisms which may cause disease, including viruses, yeasts, algae, bacteria (Gram positive and/or Gram negative), fungi, mildews, molluscs, protists (e.g. amoeba) and protozoa and prevent diseases caused by such organisms. Examples are: Thrush, Mud fever (e.g. the bacteria associated with mud fever), Ringworm (e.g. the dermatophytes that cause ringworm such as, Microsporum, Epidermophyton and Trichophyton, e.g. Tricophyton equinum), MRSA, E. coli (e.g. E. coli strain NCTC12923), Aspergillus (e.g. Aspergillus niger), Salmonella (e.g. Salmonella typhimurium), Pseudomonas (e.g. Pseudomonas aeruginosa), Streptococcus (e.g. Streptococcus pyrogenes), Listeria (e.g. Listeria sp.), Staphylococcus (e.g. Staphylococcus aureus), Clostridium difficile, Clostridium tetani, Mycobacterium tuberculosis, Bacterial meningitis, Coxiella burnetii (Q fever), Conjunctivitis (bacterial and viral), Campylobacter (gastroenteritis), Mycotoxins, Fusarium, (e.g. Fusarium proliferatum NCPF 2949), Microsporum (e.g. Microsporum equinum NCPF 0997), the causative agent of Sweetitch, Dermatophilus congolensis (a bacterial skin complaint), the causative agent of Rainscald, the causative agents of various horse skin fungal infections, protists (e.g. amoeba), Cryptosporidium, Entamoebae, Plasmodium (a malarial parasite), bacterial spores, yeasts and moulds. In one embodiment the antimicrobial polymer compositions of the invention are used to control the spread of, or prevent diseases caused by Mycotoxins such as Fusarium; or the dermatophytes that cause ringworm (e.g. Microsporum, Epidermophyton and Trichophyton).

Antimicrobial polymer compositions as herein described form a further aspect of the invention. Thus, viewed from a further aspect the invention provides an antimicrobial polymer composition, said composition comprising an antimicrobial agent and a polymer, optionally in combination with an additive. Preferred polymers, antimicrobial agents, additives, relative amounts and means for incorporation as are described above. Particularly preferred compositions according to the invention comprise a compound of formulae (I), (II), (III) or (IV) (or a polymer thereof) and polypropylene, wherein said compound of formulae (I), (II), (III) or (IV) forms from 0.1 to 5 wt %, preferably 0.25 to 1 or 2 wt % of said composition. Compounds of formula (III) or (IV) are particularly preferred in such compositions.

Use of the above-mentioned antimicrobial agents, polymers, antimicrobial polymers and compositions in the production of articles as herein described such as equestrian articles, e.g. horse grooming tools forms a further aspect of the invention. The invention thus provides a method for manufacturing an article, particularly an article for use in contact with animals such as a pet product or an equestrian article, e.g. a horse grooming tool, said method comprising forming at least part of said article from an antimicrobial agent and a polymer, preferably an antimicrobial polymer, as herein described. Preferably the article is an equestrian or pet article, e.g. a brush, sponge or comb. Particularly preferably the invention provides the use of an antimicrobial polymer in the production of an article for use with an animal, e.g. an equestrian article, wherein said antimicrobial polymer is as herein defined, e.g. it is a blend of antimicrobial agent and polymer and said antimicrobial agent and said polymer are as herein defined.

Viewed from a further aspect the invention provides the use of the antimicrobial agents, antimicrobial polymers and compositions herein described to prevent the spread of diseases, particularly those caused by the microbes mentioned herein, especially bacterial infections. Particularly preferably the invention provides an antimicrobial polymer as herein defined, for use in the prevention of disease, e.g. wherein said antimicrobial polymer is a blend of antimicrobial agent and polymer and said antimicrobial agent and said polymer are as defined herein. Thus, viewed from a further aspect, the present invention provides an antimicrobial polymer composition (or article produced therefrom) or an article comprising an antimicrobial agent and a polymer as herein described for use in the prevention of disease, e.g. that caused by the microbes mentioned herein, particularly bacterial infections, e.g. strangles. A method for preventing cross-contamination with the microbes mentioned herein or preventing disease caused by the microbes mentioned herein, e.g. bacterial infections (e.g. strangles) in an animal, e.g. a horse, also forms part of the invention, said method comprising grooming said animal with a tool, e.g. a horse grooming tool, as described herein (i.e. a tool comprising an antimicrobial agent and a polymer, e.g. a composition as herein described). A further embodiment of the present invention provides a method for preventing cross-contamination with the microbes mentioned herein or preventing disease caused by the microbes mentioned herein, e.g. bacterial infections (e.g. strangles) in an animal, e.g. a horse, said method comprising using an article as herein described in contact with said animal.

A further aspect of the present invention provides articles as herein described in which an antimicrobial agent is combined with a material other than a polymer, e.g. a natural fibre. For example, a horse grooming tool, such as a brush may comprise a coating of an antimicrobial agent as herein described on bristles which are made from or comprise natural fibres. In a further aspect the invention therefore provides an article as herein described, e.g. a horse grooming tool, comprising (e.g. particularly coated with) an antimicrobial agent as described herein.

As noted above, while the invention has thus far been described in relation to equestrian articles, it is also applicable to equipment suitable for use with, e.g. in contact with, other animals, particularly domesticated animals or those in captivity, especially livestock (e.g. cows, pigs, sheep, chickens, goats etc.) or household pets (e.g. cats, dogs, rabbits etc.). By the means described herein, articles for use in contact with other animals, particularly livestock (i.e. “livestock articles”) and household pets (i.e. “pet articles”), can also be made resistant to micro-organisms such as bacteria in accordance with the invention as herein described. Particular examples are drinking and feeding troughs, buckets and blankets for use with livestock and pet clothing, e.g. coats, collars and leads; pet grooming tools, such as brushes or combs (e.g. for cats and dogs); pet toys; pet bedding, e.g. beds, blankets, rugs or baskets; litter trays; litter scoops and feeding equipment such as bowls for food or drink. These articles form a further aspect of the invention.

In one embodiment, the article of the invention is other than a horse grooming tool.

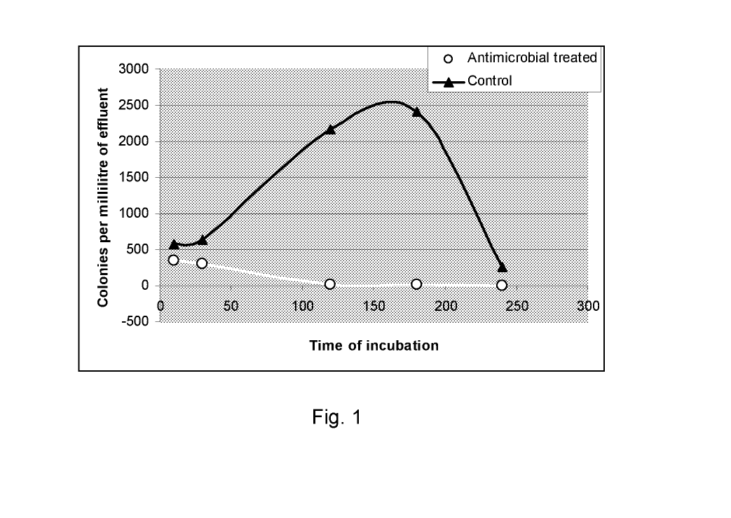

FIG. 1 shows a graphical representation of the survival of S. equi with increasing incubation times in polyprolylene fibres containing 0% (control) and 1% (active) 3-(trihydroxysilyl)propyldimethyl octodecyl ammonium chloride.

The invention will now be further described with reference to the following non-limiting Examples:

### EXAMPLE 1

### 3-(trihydroxysilyl)propyldimethyl octodecyl ammonium chloride in Polypropylene at 0.5%, 0.75% and 1.0% by Weight

3-(trihydroxysilyl)propyldimethyl octodecyl ammonium chloride (available in dry particulate form from BIOSAFE Inc., Pittsburgh, USA) was incorporated into fibres of polypropylene (molecular weight of 0.90 g/cm³) by preparing a master-batch containing 5-10% by weight of 3-(trihydroxysilyl)propyldimethyl octodecyl ammonium chloride. The master-batch was prepared by blending polyethylene resin (although polypropylene may also be used) with 3-(trihydroxysilyl)propyldimethyl octodecyl ammonium chloride in an extruder using either a single or twin screw. The recovered pellets were then blended with polypropylene resin and the melt was extruded through a die forming the filaments that were combined to form a yarn.

A ladder series using 3-(trihydroxysilyl)propyldimethyl octodecyl ammonium chloride at 0.5%, 0.75% and 1.0% by weight in the polymer fibre was prepared by the above method. Antimicrobial efficacy testing was performed to determine the optimum 3-(trihydroxysilyl)propyldimethyl octodecyl ammonium chloride loading level.

### EXAMPLE 2

### Incorporation of Antimicrobial into Polymer

The antimicrobial can be dissolved in a polar solvent (e.g. isopropyl alcohol) and added to the polymer pellets prior to processing. For example 3-(trihydroxysilyl)propyldimethyl octodecyl ammonium chloride is dissolved in an equal weight of 99.9% isopropyl alcohol. This is added to polypropylene pellets in a suitable amount, e.g. an antimicrobial solution to polymer weight ratio of 2:99 would produce a polymer containing 1% antimicrobial. The polymer pellets are stirred to incorporate the active agent evenly and then are dried under ambient conditions in order to remove the solvent (isopropyl alcohol) from the pellets. Within one or two days of drying under ambient conditions, articles can be processed from the resulting antimicrobial polymer. The methods of these Examples are applicable to other antimicrobial agents and polymers.

### EXAMPLE 3

### Incorporation of Antimicrobial Via Master-Batch Pellets

Antimicrobial is added to polymer via a concentrated master-batch pellet which is e.g. a 1:1 mixture (by weight) of a polymer (e.g. polyethylene or polypropylene) and antimicrobial. These master-batch pellets are added to the polymer from which the article is to be made in a suitable amount during the extrusion or injection moulding process.

### EXAMPLE 4

### Antimicrobial Testing—E. coli Strain NCTC12923

3-(trihydroxysilyl)propyldimethyl octodecyl ammonium chloride was combined with polypropylene stock at a dosing rate of around 1% by total weight and the mixture melted, before extruding it into fibres that can be incorporated into a finished brush.

Bundles of the dosed and non-dosed fibres were melted at 170° C. and compressed, to manufacture a number of continuous, smooth, 2 mm thick plastic sheets. The sheets were cut to create 50×50 mm area test pieces, as required by the standard test for Anti Microbial Activity ISO 22196:2007. This test is an appropriate method for assessing the surface antimicrobial activity of plastic materials when micro-organisms are placed onto the test piece.

Nutrient broth was inoculated with E. coli strain NCTC12923 and grown overnight at 37° C. This was then diluted to obtain a challenge culture of around 9.0×10⁵ bacteria per millilitre. 200 microlitres of E. coli suspension was added to each of 12 test pieces (6 dosed with 3-(trihydroxysilyl)propyldimethyl octodecyl ammonium chloride and 6 non-dosed) and covered with 50×50 mm plastic film to prevent moisture loss. The pieces were placed in a sealed box and incubated at 37° C. for 24 hours.

After incubation, the test pieces were placed in a recovery chamber with 10 ml of sterile saline and shaken vigorously to dislodge into suspension any bacteria that may be attached to the test piece.

Any living bacteria remaining within the recovery medium were counted by the spread plate method, where samples from successive tenfold dilutions of the medium were placed upon petri dishes filled with nutrient agar and the colonies counted after 24 hours of incubation at 37° C.

All non dosed test pieces showed clear growth of surviving bacteria after subculture onto nutrient agar, whereas only half of the dosed test pieces had any viable colonies.

The dosed test pieces showed a much greater decrease in living E. coli than the non dosed pieces as shown in the following table which shows the survival of E. coli on 3-(trihydroxysilyl)propyldimethyl octodecyl ammonium chloride-dosed and non-dosed test pieces expressed as colony forming units per millilitre (CFU/ml):

| Test Piece | Non-dosed | | Test Piece | Dosed | |
|---|---|---|---|---|---|
| 1 | 6950 | (6.95 × 10³) | 1 | No growth | |
| 2 | 1350 | (1.35 × 10³) | 2 | No growth | |
| 3 | 1850 | (1.85 × 10³) | 3 | 600 | (6.0 × 10²) |
| 4 | 128500 | (1.28 × 10⁵) | 4 | 600 | (6.0 × 10²) |
| 5 | 6500 | (6.50 × 10³) | 5 | No growth | |
| 6 | 170000 | (1.70 × 10⁵) | 6 | 50 | (5.0 × 10¹) |
| Mean | 52525 | (5.25 × 10⁴) | Mean | 208 | (2.08 × 10²) |

When comparing the efficacy of the two different plastics in killing the added bacteria, the mean number of E. coli surviving on the dosed test pieces was 126 times lower than on the control, non-dosed, pieces.

The results show a clear difference between the antimicrobial-treated plastic and the plastic without an additive. This demonstrates that the inherent antimicrobial activity of polypropylene feedstock, used in brush fibre manufacture without an antimicrobial additive is low, and that the kill rate of bacteria on fibre material can be effectively enhanced using an antimicrobial additive. Furthermore, the manufacturing process has been shown to successfully combine the polypropylene with an antimicrobial agent to provide an enhanced level of protection.

The antimicrobial agent is thus sufficiently stable to withstand the re-melting of the fibres when making the test pieces and the ISO 22196:2007 method has given clear and unambiguous results.

### EXAMPLE 5

### Horse Brush Manufacture

Holes are either drilled into a wooden brush back. Fibres of antimicrobial polymer prepared according to Example 1 or Example 6 are then folded around a piece of wire which is cut to size and hammered into the hole. Fibres are secured using a staple to hold the fibre in the drilled hole—the staple goes into the hole and secures into the brush back at the base of the hole.

### EXAMPLE 6

### Fibre Manufacture

Granules of 3-(trihydroxysilyl)propyldimethyl octodecyl ammonium chloride were mixed with polypropylene. The mixture was then melted and extruded to form fibres, of varying diameters from 0.1 mm to 1.2 mm. The fibres were then bundled together—each bundle has a diameter of approx 10 cm. The bundles were then cut to the lengths required to produce brush bristles, e.g. 64 or 70 mm in the fibres of a thinner diameter, and 100 mm and 170 mm length with fibres that are 0.45 mm diameter.

### EXAMPLE 7

### Antimicrobial Testing—Streptococcus equi Subsp. equi

Fibres with antimicrobial agent were compared with equivalent fibres without antimicrobial agent.

3-(trihydroxysilyl)propyldimethyl octodecyl ammonium chloride was combined with polypropylene stock at a dosing rate of around 1% by total weight and the mixture melted, before extruding it into fibres. Control fibres were produced by a method identical other than omission of the 3-(trihydroxysilyl)propyldimethyl octodecyl ammonium chloride. The fibres were 0.45 mm with an X shape cross section and 100 mm long.

A 24 hour old agar plate of Streptococcus equi subsp. equi strain number NCTC 9682, grown on Tryptone Soy Agar (TSA) was harvested using a sterile swab and transferred to 10 ml of sterile saline and mixed thoroughly. This formed the inoculum into which fibres would subsequently be immersed. The inoculum density was calculated from serial dilution and plate counts and was 3.19×10⁶ colonies per ml.

Fifteen individual fibres from each group (active and control) were taken from a bundle and immersed in the inoculum suspension for 5 seconds. The fibres were withdrawn from the suspension, excess moisture was gently shaken from the fibres and the fibres were placed in empty sterile universal tubes for a defined length of time. It was assumed that the wetted fibres would not completely dry out when placed in the empty universal tubes for incubation and that S. equi survival would be solely influenced by the characteristics of the fibre surface on which the inoculum was placed. The following table gives details of experimental incubation timing details and replication.

| | Replicates | Replicates |
|---|---|---|
| Incubation time | (active fibres) | (control fibres) |
| 10 minutes | 3 | 3 |
| 30 minutes | 3 | 3 |
| 120 minutes | 3 | 3 |
| 180 minutes | 3 | 3 |
| 240 minutes | 3 | 3 |

The purpose of the varying of incubation time was in order to determine the survival of S. equi on fibres over time following contact with an infected surface. The survival of S. equi over time would indicate the degree of risk of transmission of S. equi infection between animals in a field setting.

After incubation, the test pieces were placed in a recovery chamber with 10 ml of sterile saline and shaken vigorously to dislodge into suspension, living bacteria that may be attached to the fibres.

Any living bacteria remaining within the recovery medium were counted using the pour plate method, where samples from successive tenfold dilutions of the medium were placed upon petri dishes which were then filled with 20 ml of Tryptone Soy Agar that had stabilised at 50° C. The colonies growing on the agar were counted after 48 hours of incubation at 37° C. to determine survival and the results are shown in the following table.

| | | |
|---|---|---|
| Incubation | Antimicrobial dosed | Control |
| time | (colonies/ml) | (colonies/ml) |

| | | | | |
|---|---|---|---|---|
| 10 mins | 510 | | 1370 | |
| | 450 | | 1170 | |
| | 200 | | 930 | |
| Mean | 347 | n = 4 | 578 | n = 6 |
| 30 mins | 600 | | 3420 | |
| | 350 | | 65 | |
| | 540 | | 990 | |
| Mean | 298 | n = 5 | 639 | n = 7 |
| 120 mins | 22 | | 260 | |
| | 6 | | 16780 | |
| | 25 | | 275 | |
| Mean | 11 | n = 5 | 2164 | n = 8 |
| 180 mins | 25 | | 13600 | |
| | 15 | | ND | |
| | 5 | | 3310 | |
| Mean | 9 | n = 5 | 2416 | n = 7 |

| | | |
|---|---|---|
| 240 mins | ND (Not detected) | 872 |
| | ND | 1140 |
| | ND | 286 |

| | | | | |
|---|---|---|---|---|
| Mean | — | n = 9 | 255 | n = 9 |

As shown in the above table and FIG. 1, the survival of viable cells decreased steadily on the active fibres, declining to undetectable levels by 4 hours of incubation. By contrast, the control fibres showed increasingly high numbers of viable cells until 4 hours of incubation had elapsed, where the lowest control counts were recorded. Increases in the number of viable colonies over time on control fibres could indicate active growth of S. equi.

There is also evidence of rapid activity of the antimicrobial dosed fibres, with 99.99% of the bacteria dying in the first 10 minutes. Moreover, there is a 39.97% reduction in survival after 10 minutes relative to controls, increasing to 99.6% after 3 hours, as shown in the following table which outlines the percentage reduction of S. equi viable colonies on antimicrobial treated fibres relative to control fibres with increasing incubation times.

| | Antimicrobial | Control | Percentage |
|---|---|---|---|
| Incubation | treated (mean | (mean | reduction |
| time | colonies | colonies | relative |
| (minutes) | per ml) | per ml) | to control |
| 10 | 347 | 578 | 39.97 |
| 30 | 298 | 639 | 53.36 |
| 120 | 11 | 2164 | 99.49 |
| 180 | 9 | 2416 | 99.63 |
| 240 | 0 | 255 | 100 |

The results show a clear difference between the antimicrobial-treated polymer and the polymer without an additive. This demonstrates that the inherent antimicrobial activity of polypropylene feedstock, used in fibre manufacture without an antimicrobial additive is low, and that the kill rate of bacteria on fibre material can be effectively enhanced using an antimicrobial additive. Furthermore, the manufacturing process has been shown to successfully combine the polypropylene with the antimicrobial agent to provide an enhanced level of protection.

The results also show that the antimicrobial agent is sufficiently stable after melting of the antimicrobial to 200° C. when making the fibres.

### EXAMPLE 8

### Effects on Horses

A three year old Sec D Leaf with a lice problem was treated with Frontline® on an afternoon and then brushed with a dandy brush made according to the present invention (comprising 2% wt. 3-(trihydroxysilyl)propyldimethyl octodecyl ammonium chloride in polypropylene) the following morning. The horse's coat went from being flat and course and full of dead and dying lice, to a smooth soft coat with all lice bodies gone. No more lice were seen to appear.

A nine year old Sec A who has itching and Cushings and was being treated with Prascend. The horse was generally itchy all year round and had a lot of scurf. One side was brushed with a dandy brush made according to the present invention (comprising 2% wt. 3-(trihydroxysilyl)propyldimethyl octodecyl ammonium chloride in polypropylene). That side had a smoother, glossier coat and barely any scurf in comparison with the side that was not brushed with the brush of the invention which remained scurfy. The horse did not appear to itch on the side that has been ‘treated’ with the brush of the invention.

### EXAMPLE 9

### Effects on a Calf

A calf had the following symptoms: a raw patch on the forehead by each eye; rubbed areas up to the top of the skull where the horn buds were forming, the inside edges of the back of the ears and some bald patches on the back of the ears; a rubbed sore neck crest and rubbed patches on either side of the neck just above the shoulder. There seemed to have some scurf in her coat, believed to be due to fungus/bacteria.

The calf was brushed both days at the weekend and one evening a week with a brush according to the invention (comprising 2% wt. 3-(trihydroxysilyl)propyldimethyl octodecyl ammonium chloride in polypropylene). After eight treatments with the brush, the calf was found to be much less sore with improvement to the head areas and the incidences of scratching were greatly reduced.

### EXAMPLE 10

### Efficacy of Active Fibres and a Control Against Fusarium and Microsporum

200 millilitre cultures of both fungi (Fusarium and Microsporum) were grown and approximately 10 grams of polypropylene fibres were inserted into the cultures to determine any differences in the weight of fungal material that grows on the different fibres. Care was taken to weigh the exact mass of fibres before starting the experiment. Blue polypropylene fibres incorporated 2% wt. 3-(trihydroxysilyl)propyldimethyl octodecyl ammonium chloride and green polypropylene fibres, without an active agent, were used as a control.

After 4 days of incubation at 37° C., the cultures were harvested, the liquid was filtered away, the material on the fibres and filter was dried and the total dried material weighed. When subtracting the original weight of the fibres and the filter, any change in weight can only be the dried remains of fungal growth. The average biomass in each case is reported below.

Fusarium average biomass on green fibres=0.303 grams

Fusarium average biomass on blue fibres=0.061 grams

This shows almost five times less growth by Fusarium on the active fibres.

Microsporum average biomass on green fibres=0.980 grams

Microsporum average biomass on blue fibres=0.109 grams

This shows almost nine times less growth by Microsporum on the active fibres.

With replication, the green control fibres (i.e. those without the antimicrobial) were found to support much more biomass (or dry weight) of both fungal types than the activated (blue) ones.

Preferred embodiments of the invention include the following:

1. An equestrian article comprising an antimicrobial agent and a polymer.

2. The article as defined in embodiment 1 wherein said antimicrobial agent and said polymer are combined to form an antimicrobial polymer.

3. The article as defined in embodiment 1 or embodiment 2 comprising a blend of antimicrobial agent and polymer.

4. The article as defined in any one of the preceding embodiments wherein said article is selected from horse grooming tools (e.g. a brush, comb or sponge), equestrian horse wear, stable equipment (e.g. buckets, troughs, rugs and blankets) and riding equipment.

5. The article as defined in any one of the preceding embodiments wherein the antimicrobial agent is selected from silver compounds, biguanides, epoxy compounds and quaternary ammonium compounds.

6. The article as defined in any one of the preceding embodiments wherein the antimicrobial agent is a quaternary ammonium compound.

7. The article as defined in any one of the preceding embodiments wherein the antimicrobial agent is a compound of formula (II) or a polymer thereof

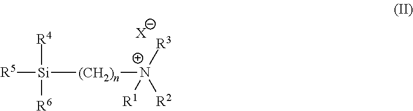

where X⁻ is a halide;
n is an integer from 2 to 6 and
R¹ to R⁶ are independently selected from an organic group, hydrogen or a hydroxy group.

8. The article as defined in embodiment 7 wherein R¹ to R⁶ are independently selected from alkyl or aryl groups, including alkoxy and aryloxy groups.

9. The article as defined in embodiment 7 wherein one or more of the R groups, especially one or more of R⁴, R⁵ and R⁶ are hydrogen or hydroxy groups.

10. The article as defined in any one embodiments 1 to 7 where said antimicrobial agent is a compound of formula (III) or a polymer thereof:

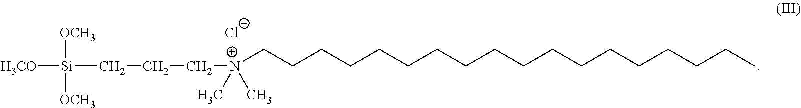 [SMILES: [Cl-] ; CCCCCCCCCCCCCCCCCC[N+](C)(C)CCC[Si](OC)(OC)OC]

11. The article as defined in any one of embodiments 1 to 7 wherein said antimicrobial agent is a compound of formula (IV) or a polymer thereof:

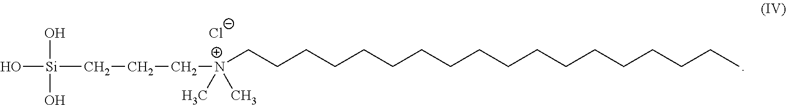 [SMILES: [Cl-] ; CCCCCCCCCCCCCCCCCC[N+](C)(C)CCC[Si](O)(O)O]

12. The article as defined in any one of embodiments 2 to 11 wherein said antimicrobial polymer is a blend of a homopolymer of antimicrobial agent and a polymer.

13. The article as defined in any one of the preceding embodiments wherein said polymer is polypropylene, or a polymer blend comprising polypropylene.

14. The article as defined in any one of embodiments 2 to 13 wherein the amount of antimicrobial agent in the antimicrobial polymer is 0.1 to 2.5 wt %.

15. The article as defined in any one of the preceding embodiments wherein said article is a textile.

16. An antimicrobial polymer composition, said composition comprising a compound of formula (III) or (IV) (or a polymer thereof) and polypropylene,

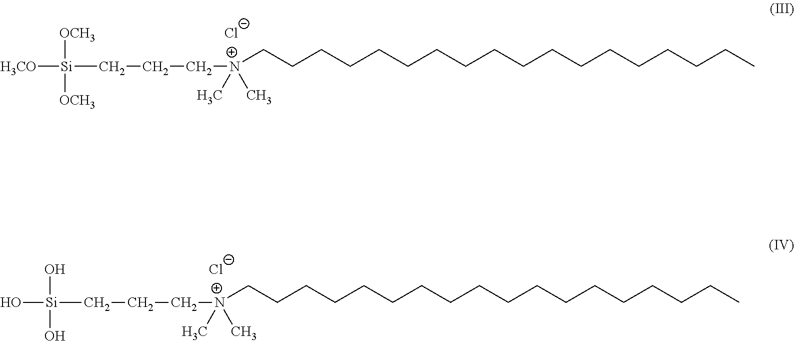 [SMILES: [Cl-] ; CCCCCCCCCCCCCCCCCC[N+](C)(C)CCC[Si](OC)(OC)OC ; [Cl-] ; CCCCCCCCCCCCCCCCCC[N+](C)(C)CCC[Si](O)(O)O]

wherein said compound of formula (III) or (IV) forms from 0.1 to 5 wt % of said composition.

17. Use of an antimicrobial polymer in the production of an equestrian article wherein said antimicrobial polymer is a blend of antimicrobial agent and polymer and said antimicrobial agent and said polymer are as defined in any one of embodiments 5 to 14.

18. An antimicrobial polymer for use in the prevention of disease, wherein said antimicrobial polymer is a blend of antimicrobial agent and polymer and said antimicrobial agent and said polymer are as defined in any one of embodiments 5 to 14.

19. The polymer as defined in embodiment 18 for use in the prevention of strangles, ringworm or diseases associated with Mycotoxins such as Fusarium.

20. A method for preventing disease in a horse, said method comprising grooming said horse with an article as defined in any one of embodiments 1 to 14.

21. The method as defined in embodiment 20 wherein said disease is strangles.

1. An article for use with an animal, particularly a domesticated animal, comprising an antimicrobial agent and a polymer, wherein the antimicrobial agent is a quaternary ammonium compound.

2. The article as claimed in claim 1, wherein said animal is a horse and said article is an equestrian article.

3. The article as claimed in claim 1, wherein said animal is a household pet or livestock.

4. The article as claimed in any one of the preceding claims wherein said antimicrobial agent and said polymer are combined to form an antimicrobial polymer.

5. The article as claimed in any one of the preceding claims comprising a blend of antimicrobial agent and polymer.

6. The article as claimed in any one of the preceding claims wherein said article is selected from horse grooming tools (e.g. a brush, comb or sponge), equestrian horse wear, stable equipment (e.g. buckets, troughs, rugs and blankets) and riding equipment.

7. The article as claimed in any one of the preceding claims wherein the antimicrobial agent is a compound of formula (II) or a polymer thereof 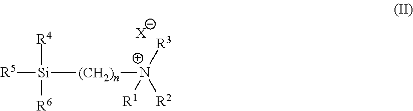
where X⁻ is a halide;
n is an integer from 2 to 6 and
R¹ to R⁶ are independently selected from an organic group, hydrogen or a hydroxy group.

8. The article as claimed in claim 7 wherein R¹ to R⁶ are independently selected from alkyl or aryl groups, including alkoxy and aryloxy groups.

9. The article as claimed in claim 7 wherein one or more of the R groups, especially one or more of R⁴, R⁵ and R⁶ are hydrogen or hydroxy groups.

10. The article as claimed in any one claims 1 to 7 where said antimicrobial agent is a compound of formula (III) or a polymer thereof:

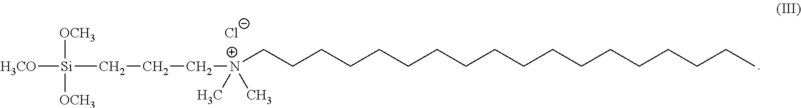 [SMILES: [Cl-] ; CCCCCCCCCCCCCCCCCC[N+](C)(C)CCC[Si](OC)(OC)OC]

11. The article as claimed in any one of claims 1 to 7 wherein said antimicrobial agent is a compound of formula (IV) or a polymer thereof:

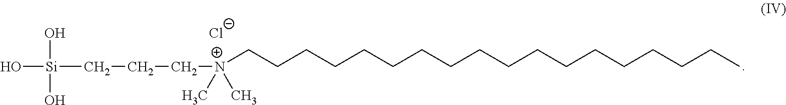 [SMILES: [Cl-] ; CCCCCCCCCCCCCCCCCC[N+](C)(C)CCC[Si](O)(O)O]

12. The article as claimed in any one of the claims 4 to 11 wherein said antimicrobial polymer is a blend of a homopolymer of said antimicrobial agent and said polymer.

13. The article as claimed in any one of the preceding claims wherein said polymer is polypropylene, or a polymer blend comprising polypropylene.

14. The article as claimed in any one of claims 4 to 13 wherein the amount of antimicrobial agent in the antimicrobial polymer is 0.1 to 2.5 wt %.

15. The article as claimed in any one of the preceding claims wherein said article is a textile.

16. An antimicrobial polymer composition, said composition comprising a compound of formula (III) or (IV) (or a polymer thereof) and polypropylene, 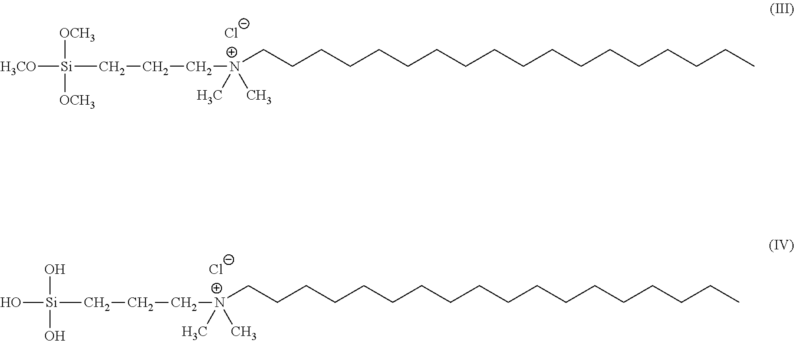 [SMILES: [Cl-] ; CCCCCCCCCCCCCCCCCC[N+](C)(C)CCC[Si](OC)(OC)OC ; [Cl-] ; CCCCCCCCCCCCCCCCCC[N+](C)(C)CCC[Si](O)(O)O]
wherein said compound of formula (III) or (IV) forms from 0.1 to 5 wt % of said composition.

17. Use of an antimicrobial polymer as defined in any one of claims 4 to 14 in the production of an article for use with an animal.

18. Use of an antimicrobial polymer in the production of an equestrian article wherein said antimicrobial polymer is a blend of antimicrobial agent and polymer and said antimicrobial agent and said polymer are as defined in any one of claims 1 to 14.

19. An antimicrobial polymer for use in the prevention of disease, wherein said antimicrobial polymer is a blend of antimicrobial agent and polymer and said antimicrobial agent and said polymer are as defined in any one of claims 1 to 14.

20. The polymer as claimed in claim 19 for use in the prevention of strangles, ringworm or diseases associated with Mycotoxins such as Fusarium.

21. A method for preventing disease in an animal (e.g. a horse), said method comprising grooming said animal (e.g. horse) with an article as claimed in any one of claims 1 to 14.

22. The method as claimed in claim 21 wherein said disease is strangles.
